(19) United States
(12) Patent Application Publication
(10) Pub. No.: US 2026/0204421 A1
(43) Pub. Date: Jul. 16, 2026

(54) ARTIFICIAL INTELLIGENCE AND WEB-BASED PLATFORM FOR DEPRESSION PREDICTION

(71) Applicant: Korea Advanced Institute of Science and Technology, Daejeon (KR)

(72) Inventors: Bum Seok Jeong, Daejeon (KR); Sunghwan Kim, Daejeon (KR); Jaewon Kim, Daejeon (KR)

(73) Assignee: Korea Advanced Institute of Science and Technology, Daejeon (KR)

(21) Appl. No.: 19/136,140

(22) PCT Filed: Nov. 17, 2023
(86) PCT No.: PCT/KR2023/018546

§ 371 Date: Jun. 5, 2025

(30) Foreign Application Priority Data
Dec. 15, 2022 (KR) 10-2022-0175465

(51) Int. Cl. G16H 50/30; A61B 5/291; G06N 20/00; G16H 10/20

(52) U.S. Cl. CPC G16H 50/30; A61B 5/291; G06N 20/00; G16H 10/20

(57) ABSTRACT

Disclosed is an artificial intelligence and web-based platform for depression prediction. Provided is a method comprising the steps of: predicting the risk of depression and suicidal idea of an examinee through an artificial intelligence model by using input data related to the examinee; and along with a result of the prediction for the risk of depression and suicidal idea, providing a feature importance value indicating the degree to which each item of the input data contributes to the prediction.

### TECHNICAL FIELD

The following description relates to a technology for predicting depression and suicidal idea.

### BACKGROUND ART

Methods for diagnosing depression may include in-depth interviews conducted by mental health professionals, self-report questionnaires, or approaches based on electroencephalogram analysis.

According to methods that predict depression based on electroencephalogram analysis, a suspected examinee may be subjected to a specific electrical stimulation to the brain, and the presence of depression can be determined by measuring the amount of the peak that occurs between 300 ms and 600 ms thereafter. However, depression diagnosis through this electroencephalogram analysis has several drawbacks, including a low signal-to-noise ratio, a complex configuration of brainwave electrodes required for precise electroencephalogram measurement, and high CPU performance requirements for processing the obtained electroencephalogram.

Furthermore, evaluating suicidal idea using structured interviews and questionnaires within clinical systems is labor-intensive and heavily dependent on clinicians, making it difficult to apply to large populations. Although there are similar technologies that predict high-risk groups for depression, anxiety, and suicide using certain data types such as video, voice, blood biomarkers, psychological assessments, and electroencephalogram, etc., they are limited in terms of the input data and model range. Moreover, these technologies do not provide real-time risk predictions to users or present the results in clearly summarized numerical values.

### PRIOR ART DOCUMENTS

### Patent Documents

Korean Patent Publication No. 10-2020-0001777 (Publication Date: Jan. 7, 2020)

### DETAILED DESCRIPTION OF THE INVENTION

### Techinical Problem

The technical problem is to provide a web-based program for predicting depression and suicidal idea, which not only classifies individuals as to whether they are experiencing depression and suicidal idea and predicts a general depression/anxiety score, but also provides a feature importance value or attention plot (or saliency map) to identify which individual input data items contribute to the prediction and which items should be examined more closely.

### Technical Solution

In a method performed in a computer system, the computer system comprises at least one processor configured to execute computer-readable instructions included in memory, and the method comprises predicting risk of depression and suicidal idea of an examinee through an artificial intelligence model by using input data related to the examinee, by the at least one processor; and providing a feature importance value indicating a degree of contribution to the prediction for each item of the input data, along with the prediction result for the risk of depression and suicidal idea.

According to an aspect, the predicting risk of depression and suicidal idea of an examinee may comprise predicting the risk of depression and suicidal idea by using a self-report questionnaire related to psychiatry, behavioral data, ecological momentary assessment (EMA), electroencephalogram (EEG), and neuroimaging data of the examinee as the input data.

According to another aspect, the predicting risk of depression and suicidal idea of an examinee may comprise predicting the risk of depression and suicidal idea by using the examinee's response to PHQ-9 which is a questionnaire for depression, GAD-7 which is a questionnaire for anxiety, STAI-X1 which is a questionnaire for state anxiety, RAS which is a questionnaire for resilience, and RSES which is a questionnaire for self-esteem.

According to another aspect, the predicting risk of depression and suicidal idea of an examinee may comprise predicting the risk of depression and suicidal idea by using a machine learning model, which is one of a support vector machine (SVM), random forest (RF), and gradient boosting, or a graph neural network, which is one of deep learning models.

According to another aspect, the predicting risk of depression and suicidal idea of an examinee may comprise predicting the risk of depression and suicidal idea by registering the input data uploaded through a web-based user interface in a queue of job scheduler, and then analyzing the input data in order of the queue.

According to another aspect, the method may further comprise calculating a general depression/anxiety score through common factor analysis based on the examinee's response to PHQ-9 which is a questionnaire for depression, GAD-7 which is a questionnaire for anxiety, and STAI-X1 which is a questionnaire for state anxiety, by the at least one processor, and providing the calculated score along with the prediction result for the risk of depression and suicidal idea.

According to another aspect, the method may further comprise predicting a general depression/anxiety score through a machine learning model based on the examinee's response to some of questionnaires consisting of PHQ-9 which is a questionnaire for depression, GAD-7 which is a questionnaire for anxiety, and STAI-X1 which is a questionnaire for state anxiety, by the at least one processor, and providing the predicted score along with the prediction result for the risk of depression and suicidal idea.

In a computer program stored in a computer-readable recording medium for executing a method for predicting depression, the method for predicting depression comprises predicting risk of depression and suicidal idea of an examinee through an artificial intelligence model by using input data related to the examinee; and providing a feature importance value indicating a degree of contribution to the prediction for each item of the input data, along with the prediction result for the risk of depression and suicidal idea.

A computer system comprises at least one processor configured to execute computer-readable instructions included in memory, and the at least one processor processes a process for predicting risk of depression and suicidal idea of an examinee through an artificial intelligence model by using input data related to the examinee; and a process for providing a feature importance value indicating a degree of contribution to the prediction for each item of the input data, along with the prediction result for the risk of depression and suicidal idea.

### ADVANTAGEOUS EFFECTS OF THE INVENTION

In clinical systems, structured interviews and questionnaires are difficult to apply to large populations. Existing related technologies have limitations in the range of input data and models, and they do not provide users with real-time risk predictions or clearly summarized numerical results. The present invention enables mental health management services in large populations by reliably and in real time providing users with predicted risk levels of depression and suicidal idea, based on various machine learning and deep learning models using diverse input information.

In addition, since coexistence of depression, anxiety disorder, and state anxiety is provided as predictions, the present invention may provide an intuitive numerical value for comprehensive severity of user's mental health status, and since a general depression/anxiety score may be predicted through a shortened questionnaire, it also may provide multimodal mental health management services.

Furthermore, by providing feature importance values for individual items that contributed to the predictions of the user's mental health status, the invention helps users understand which items are particularly relevant to their mental health risk prediction, thereby offering evidence-based materials for personalized mental health services.

### BRIEF DESCRIPTION OF THE DRAWINGS

FIG. 1 is a block diagram illustrating an example of a computer system according to an embodiment of the present invention.

FIG. 2 is a block diagram illustrating a platform system for depression and suicidal idea prediction according to an embodiment of the present invention.

FIG. 3 illustrates an example of a model for predicting risk of depression and suicidal idea according to an embodiment of the present invention.

FIG. 4 is an exemplary diagram illustrating an algorithm for calculating a general depression/anxiety score according to an embodiment of the present invention.

FIG. 5 is an exemplary diagram illustrating an algorithm for predicting a general depression/anxiety score according to an embodiment of the present invention.

FIG. 6 illustrates an example of a front-end screen on a user side of a platform for depression and suicidal idea prediction according to an embodiment of the present invention.

FIG. 7 illustrates an example of a questionnaire input screen via a web platform according to an embodiment of the present invention.

FIG. 8 is an exemplary diagram for explaining real-time input data analysis process of an analysis engine according to an embodiment of the present invention.

FIG. 9 illustrates an analysis pipeline of an analysis engine according to an embodiment of the present invention.

FIGS. 10 to 12 illustrate examples of screens for viewing results of questionnaire data analysis according to an embodiment of the present invention.

### BEST MODE FOR CARRYING OUT THE INVENTION

Hereinafter, embodiments of the present invention will be described in detail with reference to the accompanying drawings.

The embodiments of the present invention relate to a technology for predicting depression and suicidal idea.

The embodiments specifically disclosed in this specification may include classifying an individual's risk of depression and suicidal idea, calculating and predicting a general depression/anxiety score, and providing feature importance values (or attention plots) for individual items that contribute to these outcomes. The embodiments can collect responses from individual examinees through a web-based platform or upload data from large populations to evaluate mental health indicators of members in real time, and can be utilized by schools or institutions that need to manage and screen the mental health of large populations.

A platform system for depression and suicidal idea prediction according to embodiments of the present invention may be implemented by at least one computer device, and a method of providing platform for depression and suicidal idea prediction according to embodiments of the present invention may be executed through at least one computer device included in the platform system for depression and suicidal idea prediction. In this case, in the computer device, a computer program according to one embodiment of the present invention may be installed and executed, and according to the control of the executed computer program, the computer device may perform the method of providing platform for depression and suicidal idea prediction according to embodiments of the present invention. The above-described computer program may be stored in a computer-readable recording medium in conjunction with the computer device, in order to execute the method of providing platform for depression and suicidal idea prediction on the computer.

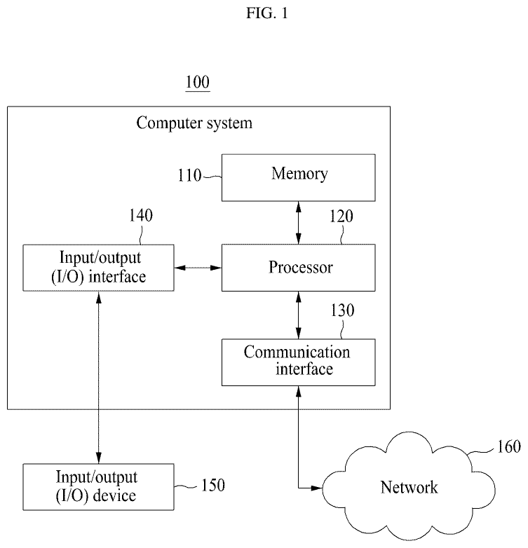

FIG. 1 is a block diagram illustrating an example of a computer system according to an embodiment of the present invention. For example, a platform system for depression and suicidal idea prediction according to embodiments of the present invention may be implemented by a computer system 100 illustrated in FIG. 1.

As shown in FIG. 1, the computer system 100 may include a memory 110, a processor 120, a communication interface 130, and an input/output (I/O) interface 140 as components for executing a method of providing platform for depression and suicidal idea prediction according to embodiments of the present invention.

The memory 110 is a computer-readable recording medium, and may include permanent mass storage devices, such as a RAM (random access memory), a ROM (read only memory) and a disk drive. Here, the permanent mass storage device, such as a ROM and a disk drive, may be included in the computer system 100 as a permanent storage device separated from the memory 110. Furthermore, an operating system and at least one program code may be stored in the memory 110. Such software components may be loaded from a computer-readable recording medium separated from the memory 110 to the memory 110. Such separate computer-readable recording medium may include computer-readable recording media, such as a floppy drive, a disk, a tape, a DVD/CD-ROM drive, a memory card, and the like. In another embodiment, software components may be loaded onto the memory 110 through the communication interface 130, not a computer-readable recording medium. For example, the software components may be loaded onto the memory 110 of the computer system 100 based on a computer program installed by files received through a network 160.

The processor 120 may be configured to process instructions of a computer program by performing basic arithmetic, logic and I/O operations. The instructions may be provided to the processor 120 by the memory 110 or the communication interface 130. For example, the processor 120 may be configured to execute instructions received according to program code stored in a recording device, such as the memory 110. The overall operations included in the method of providing platform for depression and suicidal idea prediction according to embodiments of the present invention may be performed by the processor 120.

The communication interface 130 may provide a function for enabling the computer system 100 to communicate with other devices through the network 160. For example, a request, an instruction, data or a file generated by the processor 120 of the computer system 100 according to program code stored in a recording device such as the memory 110 may be transmitted to other devices through the network 160 according to control of the communication interface 130. Inversely, a signal, an instruction, data or a file from another device may be received to the computer system 100 through the communication interface 130 of the computer system 100 passing through the network 160. A signal, an instruction or data and the like received through the communication interface 130 may be transmitted to the processor 120 or the memory 110, and a file may be stored in a storage medium (the above described permanent storage device) which may be further included in the computer system 100.

A communication method is not limited, and may include short-distance wired/wireless communication between devices in addition to communication methods using communication networks (e.g., a mobile communication network, wired Internet, wireless Internet, a broadcasting network) which may be included in the network 160. For example, the network 160 may include one or more any networks of a PAN (personal area network), a LAN (local area network), a CAN (campus area network), a MAN (metropolitan area network), a WAN (wide area network), a BBN (broadband network), and the Internet. Furthermore, the network 160 may include any one or more of network topologies, including a bus network, a star network, a ring network, a mesh network, a star-bus network, and a tree or hierarchical network, but is not limited thereto.

The I/O interface 140 may be means for interface with an input/output (I/O) device 150. For example, the input device may include a device such as a microphone, a keyboard, a camera, or a mouse and the like, and the output device may include a device such as a display or a speaker. For another example, the I/O interface 140 may be means for interface with a device in which functions for input and output have been integrated into one, such as a touch screen. The I/O device 150, together with the computer system 100, may be configured as a single device.

Furthermore, in other embodiments, the computer system 100 may include components less or more than the components of FIG. 1. However, it is not necessary to clearly illustrate most of conventional components. For example, the computer system 100 may be implemented to include at least some of the I/O device 150 above described or may further include other components such as a transceiver, a camera, various sensors, a database, etc.

The following describes specific embodiments of the artificial intelligence and web-based platform system for depression prediction and the method for providing the same.

The embodiments relate to a web-based program for predicting depression and suicidal idea in adults. There are similar technologies that predict high-risk groups for depression, anxiety, and suicide using certain data types such as video, voice, blood biomarkers, psychological assessments, and electroencephalogram, etc. The present program can utilize various types of a self-report questionnaire related to psychiatry, behavioral data, ecological momentary assessment (EMA), electroencephalogram (EEG), and neuroimaging data of an examinee as the input data. Based on these input data, the program applies machine learning models such as support vector machines (SVM), random forests (RF), and gradient boosting, as well as deep learning models with enhanced performance, such as graph neural networks, to generate predictions on the presence of depressive episodes and suicidal idea. These predictions, along with information on the models used, can be provided to health administrators responsible for managing the mental health of large adult populations.

Unlike conventional technologies, the present platform enables not only the prediction of depression and suicidal idea levels, but also the calculation of a machine learning-based general depression/anxiety score (G-DA score). This allows the severity of mental health status to be quantified in a summarized numerical format. Furthermore, it provides the contribution level of each input data item used for the prediction—such as classification of an individual's depression/suicidal idea and prediction of a general depression/anxiety score—in the form of feature importance values (or attention plots). This helps information users identify which specific items should be closely examined for each subject. In other words, the goal is to support clinical decision-making in mental health through the implementation of explainable artificial intelligence.

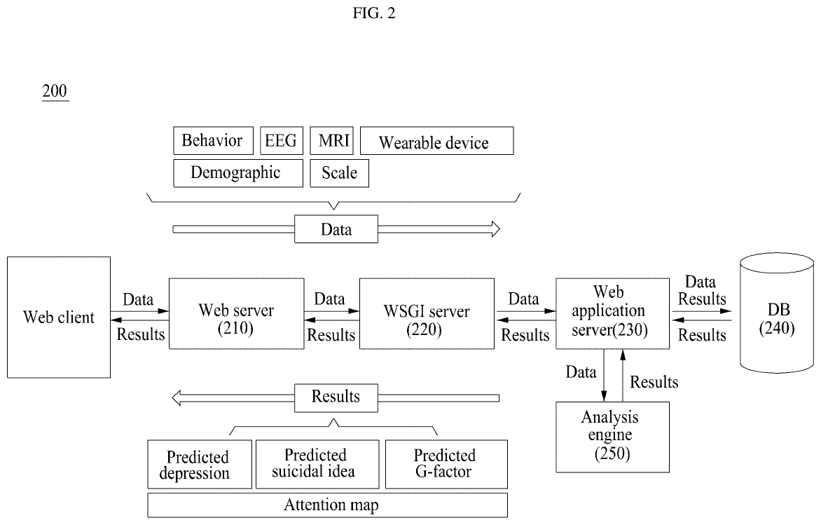

FIG. 2 is a block diagram illustrating a platform system for depression and suicidal idea prediction according to an embodiment of the present invention.

A platform system for depression and suicidal idea prediction 100 can be broadly divided into a data collection part, a data analysis part, and a results output part.

Referring to FIG. 2, users who access the web (web clients) may input data on each participant or, in the case of health administrators, may upload large-scale response data.

As the data inputted/uploaded for use in depression and suicidal idea prediction, demographic information such as gender and age, questionnaire scales, brain magnetic resonance imaging (MRI) and EEG information, daily life information from wearable devices, and behavioral data obtained through task performance, and the like are transmitted to a web application server 230 via a web server 210 and a WSGI (Web Server Gateway Interface) server 220.

The web application server 230 stores the received information in a database (DB) 240 capable of storing and retrieving large-scale data and simultaneously transmits it to an analysis engine 250 to obtain predictions for depression and suicidal idea.

The analysis engine 250 can calculate result data such as depression and suicidal idea, a general depression/anxiety score, and feature importance through pre-trained machine learning/deep learning models. The data analysis process may take from several minutes to several hours depending on the complexity of the preprocessing and the analysis engine 250, and the results obtained from the analysis are stored in the DB 240. The analysis results stored in the DB 240 can be displayed on the platform's user interface via the web application server 230, the WSGI server 220, and the web server 210 upon user's request.

For example, the web server 210 may be implemented using Nginx, the WSGI server 220 using Gunicorn, the web application server 230 using Django, and the DB 240 using MariaDB to handle multiple user requests. The analysis engine 250 may be implemented using python-based sklearn and pytorch packages.

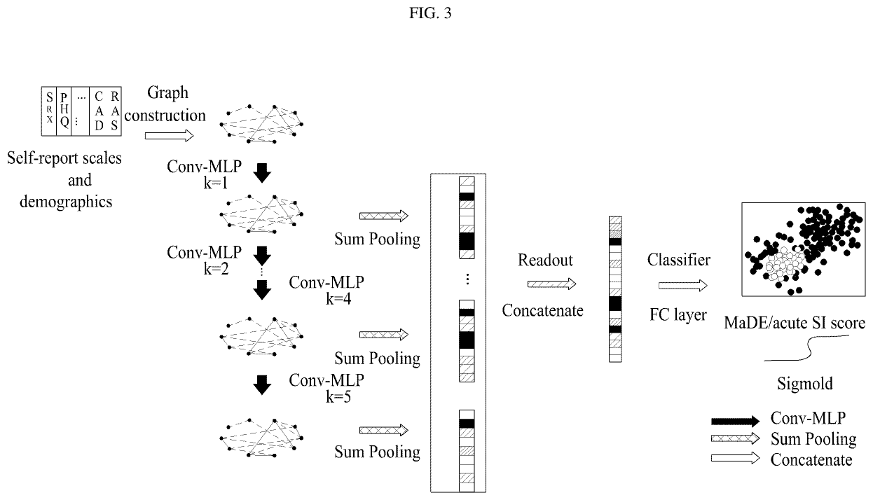

After users log into the platform system for depression and suicidal idea prediction 100, users can upload various information such as ID, test location, age, gender, and questionnaire scores. Once the analysis engine 250 completes processing and generates the analysis results, the results can be viewed using various criteria such as ID, depression risk (MDD), and suicidal idea risk. The depression risk and suicidal idea risk can be obtained by loading machine learning or deep learning model whose model parameters have been optimized based on existing data to predict the depression risk and the suicidal idea risk using the user's demographic information, such as age and gender, and individual questionnaire item scores as input. For machine learning models, pre-implemented models such as Support Vector Machine (SVM), Random Forest (RF), and Gradient Boosting can be used, and for deep learning, a graph isomorphism network (GIN), which is a type of graph neural network, can be utilized. In the case of deep learning models, the MGM (Mixed Graphical Model) package in R is used to first obtain a relational graph between age, gender, and individual questionnaire scores. The MGM package implements an algorithm that estimates relational graphs among variables from observed data. This graph is then used as the adjacency matrix for the GIN, which performs graph convolution operations across three hidden layers. The hidden variables of each hidden layer obtained from graph convolution operations are concatenated into a single vector, which is then fed into a fully connected layer classifier along with the age, gender, and individual questionnaire item scores to generate predictions of depression risk and suicidal idea (see FIG. 3). Also, on the depression analysis page, not only the overall levels of depression and suicidal idea and the general depression/anxiety score but also the key factors that were important in predicting depression and suicidal idea can be confirmed. The importance is calculated using the LIME (Local Interpretable Model-agnostic Explanations) algorithm in the case of machine learning models, and a custom-implemented graph saliency calculation algorithm in the case of deep learning models. These methods compute the degree to which predictions change when each individual input feature is altered, thereby determining the importance of all input features.

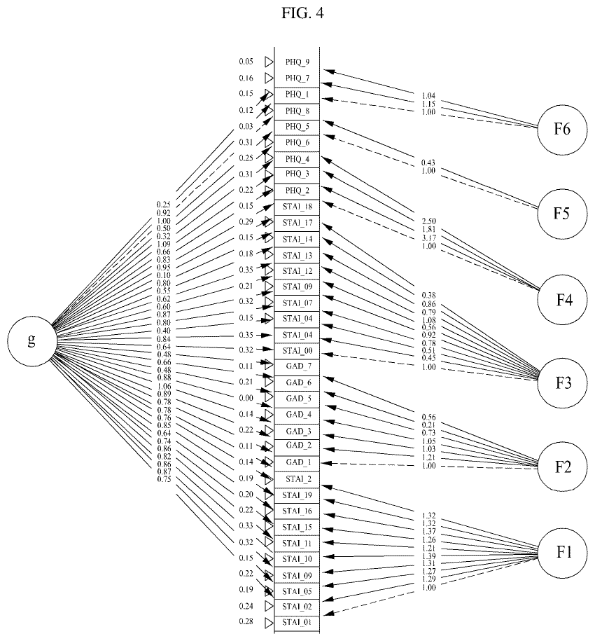

FIG. 4 is an exemplary diagram illustrating an algorithm for calculating a general depression/anxiety score according to an embodiment of the present invention.

In the present invention, a general depression/anxiety score (G-DA score) calculation algorithm measures depression, anxiety, and state anxiety—which are highly likely to coexist and therefore difficult to measure independently—as a single scalar value. Referring to FIG. 4, the processor 120 may derive the G-DA score through common factor analysis implemented in Psych package in R, for a total of 36 items consisting of PHQ-9 which is a depression scale, GAD-7 which is an anxiety scale, and STAI-X1 which is a state anxiety. The common factor analysis is a statistical method that identifies a small number of latent variables (factors) that can group similar items (common dimensions) based on common variance among various collected variables, and the Psych package is a package which enables this statistical method to be applied in R. During the common factor analysis, a single latent common factor is assumed for the common variance, and a loading matrix is obtained to reflect how well each variable is explained by this factor. Once the loading matrix is obtained, the common latent factor value for each input dataset can be calculated. In this invention, the value of the common factor obtained by inputting the depression, generalized anxiety, and state anxiety scales is considered the general depression/anxiety score (G-DA score). After obtaining a G-DA score for each user's data via common factor analysis, supervised learning can be applied to predict the G-DA score using input data. By using a model trained to estimate the G-DA score, it is possible to obtain a predicted G-DA score when a new user inputs their depression, generalized anxiety, and state anxiety scale values. The input features that are important for the prediction can be identified using the same importance measurement algorithms described earlier.

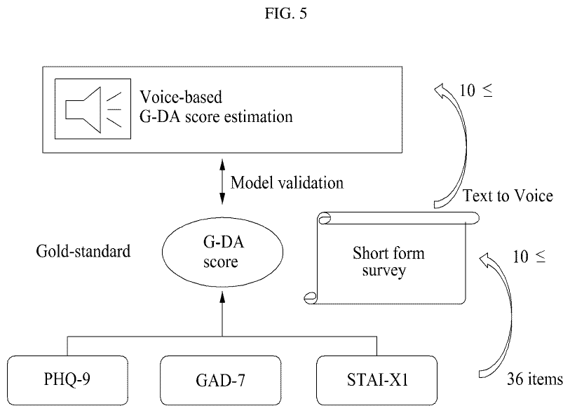

FIG. 5 is an exemplary diagram illustrating an algorithm for predicting a general depression/anxiety score according to an embodiment of the present invention.

In the present invention, a general depression/anxiety score (G-DA score) prediction algorithm predicts the G-DA score with high reliability using only some of 36 items consisting of PHQ-9, GAD-7, and STAI-X1. The reliability of the G-DA score can be confirmed by calculating the mean absolute error or mean squared error between the predicted value—obtained using some of items—and the actual G-DA score, and demonstrating that this error is nearly identical to the error of a model using the full of items. Referring to FIG. 5, the processor 120 can predict the G-DA score using a reduced number of items (e.g., fewer than 10 items of the 36 items) from the 36 items consisting of PHQ-9, GAD-7, and STAI-X1, thereby preventing user drop-out in situations where repeated daily measurements are taken or in voice-based environments, and increasing reliability of the test results.

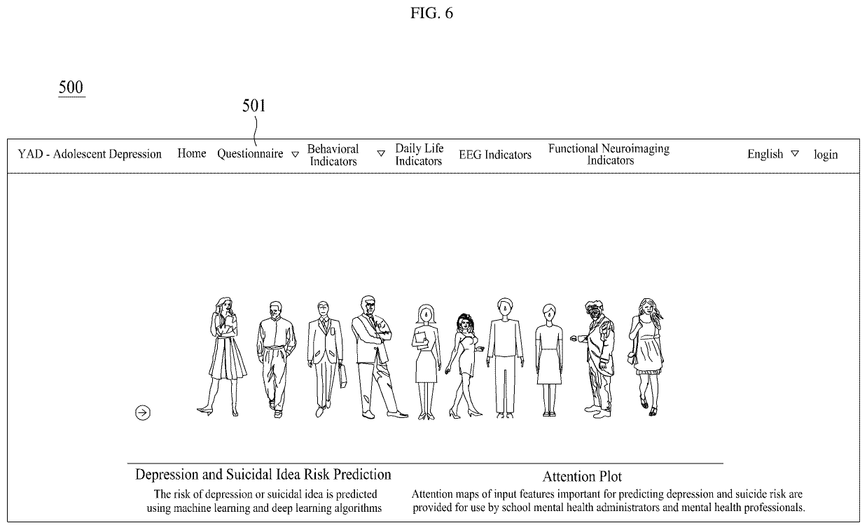

FIG. 6 illustrates an example of a front-end screen on a user side of a platform for depression and suicidal idea prediction according to an embodiment of the present invention.

FIG. 6 shows a home screen 500 of a platform for depression and suicidal idea prediction. For example, a web-based user interface of the platform for depression and suicidal idea prediction may be implemented as shown in FIG. 6. Because mental health screening data may be sensitive to security, access is restricted to authorized users through a login process. The home screen 500 of the platform for depression and suicidal idea prediction, as a web-based user interface, may provide a menu 501 for questionnaire input as one of the menus on the web.

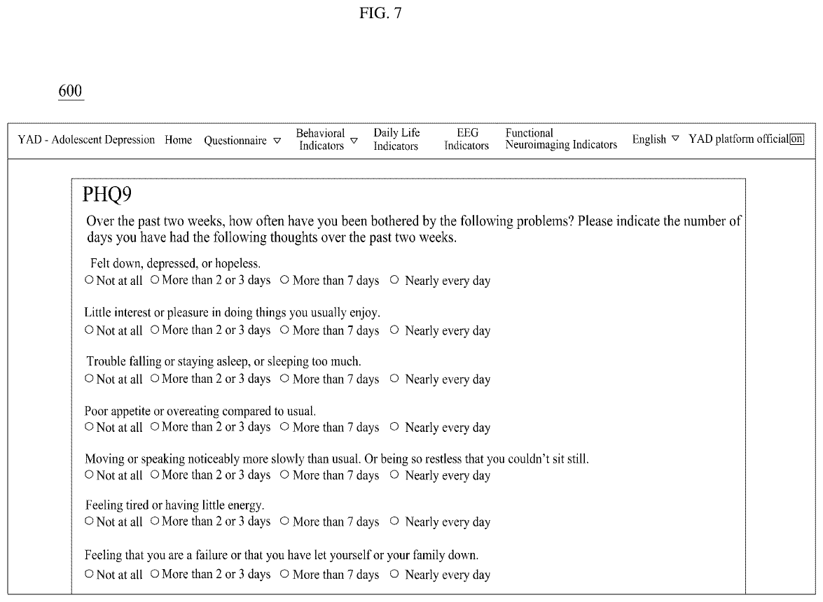

FIG. 7 illustrates an example of a questionnaire input screen via a web platform according to an embodiment of the present invention.

The processor 120 may provide a questionnaire input screen 600 when a user logs in to the home screen 500 of the platform for depression and suicidal idea prediction and selects the ‘Questionnaire Input’ menu 501.

Referring to FIG. 7, if the user wishes to complete a questionnaire through the web platform, i.e., the platform system for depression and suicidal idea prediction 100, the user may select the ‘Questionnaire Input’ menu 501 of the menus on the web and enter responses to key mental health status-related questionnaires through the questionnaire input screen 600. For example, the questionnaire may include 1) 9 items related to depression (PHQ-9), 2) 7 items related to anxiety disorder (GAD-7), 3) 20 items related to state anxiety (STAI-X1), 4) 10 items related to resilience (RAS), 5) 12 items related to self-esteem (RSES).

In the platform for depression and suicidal idea prediction according to the present invention, a questionnaire data file upload environment may be provided to allow massive data to be uploaded at once when health administrators or similar users need to analyze large-scale mental health screening results.

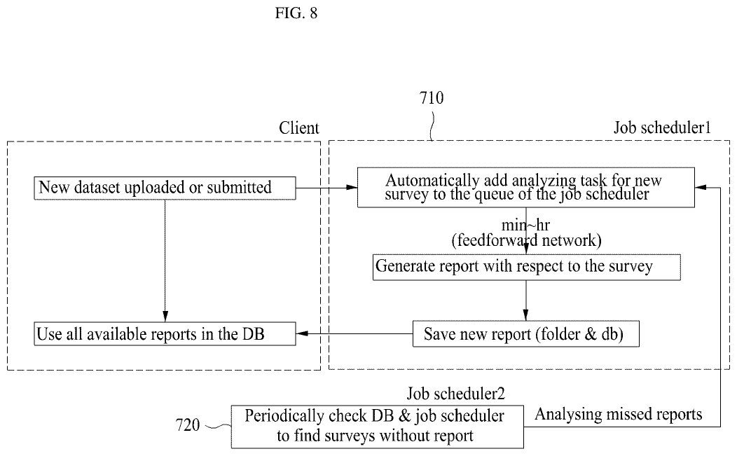

FIG. 8 is an exemplary diagram for explaining real-time input data analysis process of an analysis engine according to an embodiment of the present invention.

The platform for depression and suicidal idea prediction according to the present invention may include a process for delivering analysis results via the web in the shortest possible time by analyzing input data in real-time as one of its core elements.

Referring to FIG. 8, the analysis engine 250 may include a job scheduling process, and a first job scheduler 710 registers newly submitted or uploaded data for analysis by a user (client) into the job scheduler queue immediately and begins analysis in the order of the queue. When using a pre-trained model, the result generation may take several minutes for questionnaire data and several hours for neuroimaging data. Once the analysis is completed, data is generated as a report including the analysis results and stored in the DB 240. The report stored in the DB 240 can be displayed via the result viewing screen of the platform.

A second job scheduler 720 periodically checks the input data and adds input data that does not produce analysis results and does not exist in the queue to the analysis queue. The analysis engine 250 can manage the process via the second job scheduler 720 to ensure that no input data is excluded from analysis.

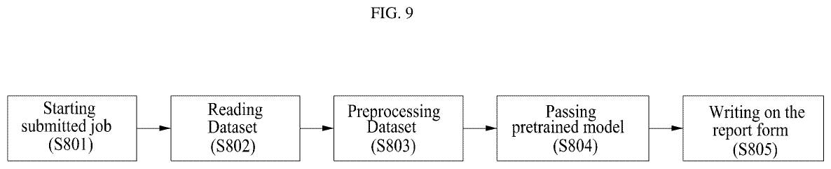

FIG. 9 illustrates an analysis pipeline of an analysis engine according to an embodiment of the present invention.

Referring to FIG. 9, when a task is submitted by the first job scheduler 710 S801, the analysis engine 250 stores the data in data directory and the DB 240, then reads it S802 and performs data preprocessing in a format required by a prediction model S803. At this time, the analysis engine 250 may also store the preprocessing results for later use in data analysis quality checks. The analysis engine 250 may predict risk of depression/suicidal idea and calculate a general depression/anxiety score (G-DA score) using pre-trained machine learning/deep learning models S804. The analysis engine 250 generates a report including the predicted results for risk of depression/suicidal idea and the G-DA score, and stores it in the data directory and the DB 240 S805.

For categorical variable prediction such as the risk of depression and suicidal idea, the analysis engine 250 may use machine learning models such as Support Vector Machine (SVM) or Random Forest (RF). For continuous variable prediction such as the general depression/anxiety score (G-DA score), it may use machine learning models such as Light Gradient Boosting (LGB).

In another example, deep learning models such as Graph Neural Networks (GNN) may be used for predicting depression and suicidal idea. By employing deep learning architectures such as GNN, performance for predicting depression and suicidal idea based on questionnaire responses or resting-state functional brain connectivity can be improved.

The analysis engine 250 may store user information, questionnaire responses, and questionnaire analysis results in the DB 240, and the DB 240 may use a MySQL-based system such as MariaDB.

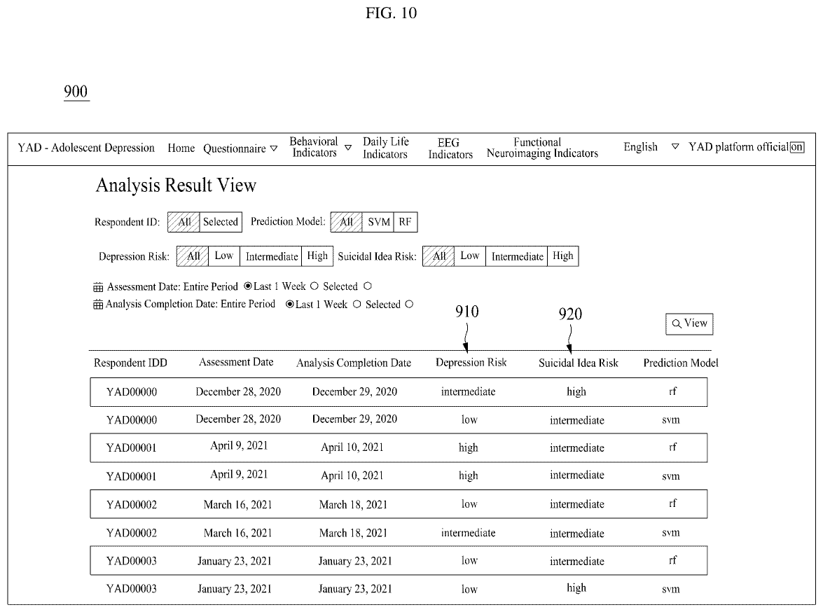

FIGS. 10 to 12 illustrate examples of screens for viewing results of questionnaire data analysis according to an embodiment of the present invention.

The processor 120 may provide analysis results through a result viewing screen when the analysis of the user's input data is completed. FIG. 10 illustrates an analysis result viewing screen 900.

The processor 120 may predict risk of depression and suicidal idea using two models and generate two prediction result reports for a single individual.

The processor 120 may provide predicted results of depression risk 910 and predicted results of suicidal idea risk 920 for each questionnaire respondent through the analysis result viewing screen 900.

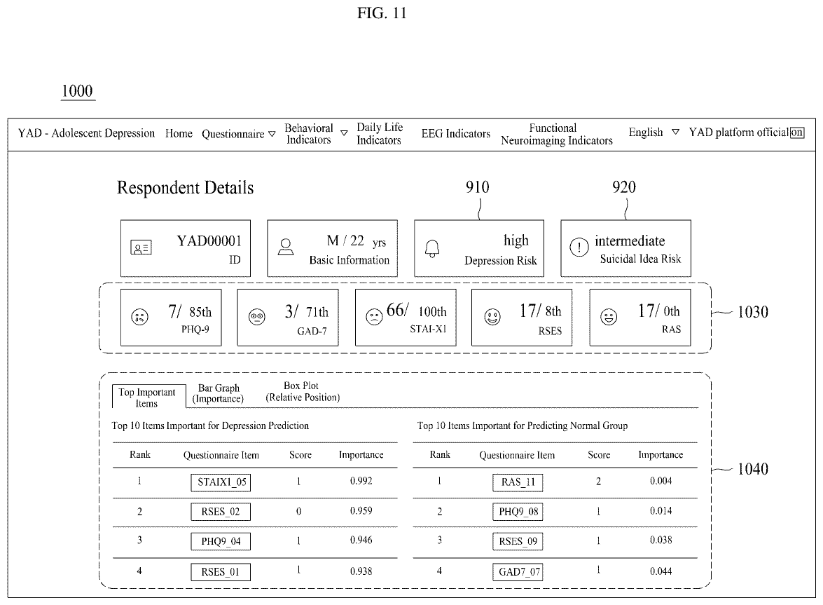

When a specific questionnaire respondent is selected from the analysis result viewing screen 900, the processor 120 may provide a detailed analysis result screen 1000 including detailed information of the selected respondent, as shown in FIG. 11.

Referring to FIG. 11, the processor 120 may display the predicted results of depression risk 910 and the predicted results of suicidal idea risk 920, along with the respondent's identification number and basic information, through the detailed analysis result screen 1000. Furthermore, the processor 120 may summarize and display relative position percentile rank 1030 based on the total score for each questionnaire type (PHQ-9, GAD-7, STAI-X1, RSES, RAS) through the detailed analysis result screen 1000. In addition, the processor 120 may identify and display the top 10 items that were most important (i.e., received the highest attention) in predicting the risk of depression and suicidal idea as top important item information 1040 through the detailed analysis result screen 1000.

The processor 120 may provide a prediction of the general depression/anxiety score as part of the analysis results for the respondent through the detailed analysis result screen 1000, and may also provide information on the models used for the prediction of the depression/suicidal idea risk and the prediction of the general depression/anxiety score.

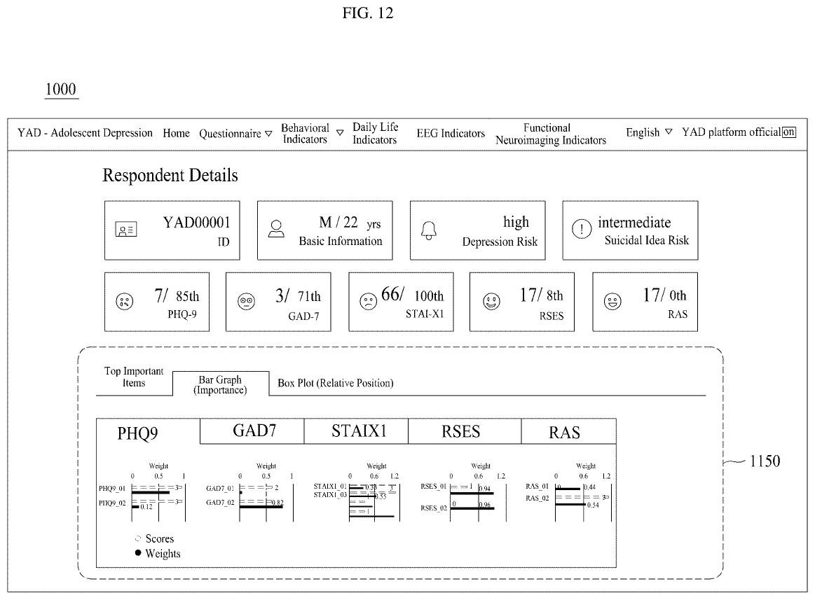

Referring to FIG. 12, the processor 120 may provide, through the detailed analysis result screen 1000, not only the top important item information 1040 but also overall item information 1150, which visualizes in a bar graph the prediction importance (attention) of all items used for predicting risk of depression and suicidal idea for each questionnaire type (PHQ-9, GAD-7, STAI-X1, RSES, RAS) in a comprehensive and organized manner.

Accordingly, the platform system for depression and suicidal idea prediction according to embodiments of the present invention can receive various types of information as input such as questionnaire, behavioral data, ecological momentary assessment (EMA), electroencephalogram (EEG), and neuroimaging data in order to overcome the limitations of conventional technologies for depression prediction. It provides users with predictions of the depression and suicidal idea risk, as well as predictions of the general depression/anxiety score, using a variety of machine learning and deep learning models. In addition, in the present embodiment, the feature importance values of individual items are provided so that users can understand which items they should pay attention to in relation to their personal mental health risk predictions. Furthermore, in addition to specific mental health indicators limited to individual areas such as depression and suicidal idea, predictions for the general depression/anxiety score which provides a compressed overview of overall mental health status may also be provided.

As such, according to the embodiments of the present invention, by providing a web-based platform that predicts depression and suicide risk in real time, the service can be applied to large-scale populations, and when using pre-trained machine learning/deep learning models, it can provide rapid predictions and attention plots for a large volume of input data. That is, the web-based platform for depression and suicidal idea prediction allows for the broad provision of services with minimal maintenance cost, can be extended to various mental health issues beyond depression, and can be easily applied in industrial contexts such as schools and institutions.

The aforementioned device may be implemented as a hardware component, a software component, and/or a combination of a hardware component and a software component. For example, the device and component described in the embodiments may be implemented using one or more general-purpose computers or special-purpose computers, such as a processor, a controller, an arithmetic logic unit (ALU), a digital signal processor, a microcomputer, a field programmable gate array (FPGA), a programmable logic unit (PLU), a microprocessor, or any other device capable of executing or responding to an instruction. The processing device may perform an operating system (OS) and one or more software applications that are executed on the OS. Furthermore, the processing device may access, store, manipulate, process, and generate data in response to the execution of software. For convenience of understanding, one processing device has been illustrated as being used, but a person having ordinary knowledge in the art may understand that the processing device may include a plurality of processing elements and/or a plurality of types of processing elements. For example, the processing device may include a plurality of processors or one processor and one controller. Furthermore, another processing configuration, such as a parallel processor, is also possible.

Software may include a computer program, a code, an instruction or a combination of one or more of them, and may configure a processing device so that the processing device operates as desired or may instruct the processing devices independently or collectively. The software and/or the data may be embodied in any type of machine, a component, a physical device, virtual equipment, or a computer storage medium or device in order to be interpreted by the processing device or to provide an instruction or data to the processing device. The software may be distributed to computer systems that are connected over a network, and may be stored or executed in a distributed manner. The software and the data may be stored in one or more computer-readable recording media.

The method according to an embodiment may be implemented in the form of a program instruction executable by various computer means and stored in a computer-readable medium. The medium may continue to store a program executable by a computer or may temporarily store the program for execution or download. Furthermore, the medium may be various recording means or storage means of a form in which one or a plurality of pieces of hardware has been combined. The medium is not limited to a medium directly connected to a computer system, but may be one distributed over a network. Examples of the medium may be magnetic media such as a hard disk, a floppy disk and a magnetic tape, optical media such as a CD-ROM and a DVD, magneto-optical media such as a floptical disk, and media configured to store program instructions, including, a ROM, a RAM, and a flash memory. Furthermore, other examples of the medium may include an app store in which apps are distributed, a site in which various pieces of other software are supplied or distributed, and recording media and/or storage media managed in a server.

### DESCRIPTION OF EMBODIMENTS

As described above, although the embodiments have been described in connection with the limited embodiments and the drawings, those skilled in the art may modify and change the embodiments in various ways from the description. For example, proper results may be achieved although the aforementioned descriptions are performed in order different from that of the described method and/or the aforementioned components, such as a system, a structure, a device, and a circuit, are coupled or combined in a form different from that of the described method or replaced or substituted with other components or equivalents thereof.

Accordingly, other implementations, other embodiments, and the equivalents of the claims fall within the scope of the claims.

1. A method performed in a computer system, wherein the computer system comprises at least one processor configured to execute computer-readable instructions included in memory, and
the method comprises:
predicting risk of depression and suicidal idea of an examinee through an artificial intelligence model by using input data related to the examinee, by the at least one processor; and
providing a feature importance value indicating a degree of contribution to the prediction for each item of the input data, along with the prediction result for the risk of depression and suicidal idea.

2. The method of claim 1, wherein the predicting risk of depression and suicidal idea of an examinee comprises predicting the risk of depression and suicidal idea by using a self-report questionnaire related to psychiatry, behavioral data, ecological momentary assessment (EMA), electroencephalogram (EEG), and neuroimaging data of the examinee as the input data.

3. The method of claim 1, wherein the predicting risk of depression and suicidal idea of an examinee comprises calculating a general depression/anxiety score through common factor analysis based on the examinee's response to PHQ-9 which is a questionnaire for depression, GAD-7 which is a questionnaire for anxiety, and STAI-X1 which is a questionnaire for state anxiety, by the at least one processor, and providing the calculated score along with the prediction result for the risk of depression and suicidal idea.

4. The method of claim 1, wherein the predicting risk of depression and suicidal idea of an examinee comprises predicting the risk of depression and suicidal idea by using a machine learning model, which is one of a support vector machine (SVM), random forest (RF), and gradient boosting, or a graph neural network, which is one of deep learning models.

5. The method of claim 1, wherein the predicting risk of depression and suicidal idea of an examinee comprises predicting the risk of depression and suicidal idea by registering the input data uploaded through a web-based user interface in a queue of job scheduler, and then analyzing the input data in order of the queue.

6. The method of claim 1, wherein the method further comprises calculating a general depression/anxiety score through common factor analysis based on the examinee's response to PHQ-9 which is a questionnaire for depression, GAD-7 which is a questionnaire for anxiety, and STAI-X1 which is a questionnaire for state anxiety, by the at least one processor, and providing the calculated score along with the prediction result for the risk of depression and suicidal idea.

7. The method of claim 1, wherein the method further comprises predicting a general depression/anxiety score through a machine learning model based on the examinee's response to some of questionnaires consisting of PHQ-9 which is a questionnaire for depression, GAD-7 which is a questionnaire for anxiety, and STAI-X1 which is a questionnaire for state anxiety, by the at least one processor, and providing the predicted score along with the prediction result for the risk of depression and suicidal idea.

8. (canceled)

9. A computer system, comprising at least one processor configured to execute computer-readable instructions included in memory, wherein the at least one processor processes:
a process for predicting risk of depression and suicidal idea of an examinee through an artificial intelligence model by using input data related to the examinee; and
a process for providing a feature importance value indicating a degree of contribution to the prediction for each item of the input data, along with the prediction result for the risk of depression and suicidal idea.

10. The computer system of claim 9, wherein the at least one processor is configured to calculate a general depression/anxiety score based on the examinee's response to at least some of questionnaires consisting of PHQ-9 which is a questionnaire for depression, GAD-7 which is a questionnaire for anxiety, and STAI-X1 which is a questionnaire for state anxiety, and providing the calculated score along with the prediction result for the risk of depression and suicidal idea.
